(19) United States
(12) Patent Application Publication
(10) Pub. No.: US 2004/0086572 A1
(43) Pub. Date: May 6, 2004

(54) Delivery of therapeutic agent affixed to magnetic particle

(76) Inventors: James P. Dailey, Erie, PA (US); Judy Riffle, Blacksburg, VA (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/620,762

(22) Filed: Jul. 17, 2003

Related U.S. Application Data
(60) Provisional application No. 60/396,085, filed on Jul. 17, 2002.
(60) Provisional application No. 60/444,960, filed on Feb. 5, 2003.

(51) Int. Cl. A61K 39/395; A61K 31/56; A61K 33/26; A61K 9/14

(52) U.S. Cl. 424/489; 424/145.1; 424/646; 514/171

(57) ABSTRACT

A method for targeted delivery of therapeutic agents within the eye is provided. Magnetic particles having associated therapeutic agents are injected into the eye, for example into the vitreous cavity. External magnets are then used move the particles to a desired position within the eye, for example, to the macula. The therapeutic agent that is delivered may be, for example, anti-VEGF (for the treatment of exudative macular degeneration) or a steroid (for the treatment of diabetic retinopathy).

### BACKGROUND OF THE INVENTION

1. Field of the Invention

The invention generally relates to the targeted delivery of therapeutic agents within the eye. In particular, the invention provides methods for targeted delivery to the macula of therapeutic agents which are attached to magnetic particles.

2. Background of the Invention

In the past several years, a number of ocular disease processes have been treated with increasing frequency via intravitreal injection. Intravitreal injection involves the injection of drug via fine needle under local (eyedrop) anesthesia, into the vitreous cavity of the eye. When properly done, it is safe and relatively painless. Intravitreal injections are currently used to treat ocular inflammatory diseases, such as posterior uveitis and cystoid macular edema (usually from inflammation or following cataract surgery). There are promising ongoing clinical studies looking at intravitreal injection of steroid for the treatment of clinically significant macular edema from diabetic retinopathy, and intravitreal injection of anti-angiogenic agents for the treatment of exudative macular degeneration. An advantage of intravitreal injection is that it provides a higher concentration of drug within the eye (i.e., in the general area of pathology) than any of the other available routes, which include topical (eyedrops), systemic (intravenous and oral administration), and extraocular (subtenon's) injection. However, a disadvantage of intravitreal injection is that the delivery is not specifically directed to the macula, which is the precise target for treatment.

The prior art has thus far failed to provide methods for delivery of and/or concentration of therapeutic agents in the eye specifically at the macula.

### SUMMARY OF THE INVENTION

The present invention provides a method of positioning or concentrating a therapeutic agent within the eye, for example, at the macula. The therapeutic agent is affixed to magnetic particles and injected into the eye. Using external magnets according to the methods of the present invention, the magnetic particles are selectively positioned within the eye, thus providing a high concentration of the therapeutic agent at a desired location.

It is an object of this invention to provide a method of delivering a therapeutic agent to a desired location within an eye. The method comprises the steps of providing to the eye a formulation comprising magnetic particles. The magnetic particles have at least one associated therapeutic agent. A magnetic field is used to move at least a portion of the magnetic particles to the desired location within the eye. The particles may be provided by injecting the formulation into the vitreous cavity of the eye. The desired location may be the macula of the eye.

The therapeutic agent may be, for example, anti-VEGF or a steroid. The magnetic particles may be made from a material such as cobalt, magnetite, or nickel. The particles may be nanoparticles or microparticles.

In one embodiment of the invention, the portion of particles reaching the desired location is greater than 50% of the magnetic particles provided in the providing step.

The magnetic particles may have one associated therapeutic agent. Alternatively, the magnetic particles may have more than one associated therapeutic agent. Further, a label may be associated with the magnetic particles.

### BRIEF DESCRIPTION OF THE DRAWINGS

FIG. 1. Schematic depiction of the method of the invention. Cross sectional view of an eye in which magnetic particles with attached therapeutic agent(s) are injected into the vitreous cavity, and the particles are driven by an external magnet to a target site in the eye.

FIG. 2 illustrates the placements of an external magnet relative to the eye of the patient in order to create a suitable magnetic field.

### DETAILED DESCRIPTION OF THE PREFERRED EMBODIMENTS OF THE INVENTION

The present invention provides a method of positioning or concentrating a therapeutic agent within the eye, for example, at the macula. The therapeutic agent is affixed to magnetic particles and is injected into the vitreous cavity of the eye. External magnets are used to generate a magnetic field and to selectively position the magnetic particles within the eye, thus allowing concentration of the therapeutic agent at the desired location.

The possible clinical use of magnetically guided particles for drug delivery to tumors and elsewhere within the body has been studied for many years. There is a vast literature on the topic [Häfeli et al., 1997; Lübbe et al., 2001; \check{S}afa\check{r}ik and \check{S}afa\check{r}ková 2002] and ongoing commercial development of various methods and materials for use in such therapies, eg. [U.S. Pat. No. 6,200,547 to Volkonsky et al.; U.S. Pat. Ser. No. 6,482,436 to Volkonsky et al; U.S. Pat. Ser. No. 6,488,615 to Mitchiner], is taking place. In parallel with this work has been the study of magnetic concentration of micro- and nanoparticles for more biomechanically-oriented purposes. For example, the thrombosis and occlusion of intracranial aneurysms is discussed by Gillies et al [Gillies et al, 1994]) and Dailey et al [Dailey et al, 1997; Dailey et al., 1999], describe employing magnetic pressure to augment the function of intraocular silicone fluid tamponades used for the repair of retinal detachments. See U.S. Pat. Ser. No. 6,135,118 to Dailey, the complete contents of which are hereby incorporated by reference.

The present invention involves the novel application of magnetic particle technology to the treatment of ocular disorders. According to the practice of the present invention, a therapeutic agent is affixed to a magnetic particle. A solution of such magnetic particles is injected into the vitreous cavity of the eye of a patient. Those of skill in the art will recognize that the composition of the vitreous cavity of adults (i.e. after about age 18) is largely aqueous in nature, and that it is possible for particulate matter to move through the aqueous medium of the cavity relatively unhindered if a proper force is applied. In the practice of the present invention, a suitable magnetic force is applied to the particles by arranging at least one magnet either 1) externally and temporarily (e.g. behind the head) or 2) internally and permanently (e.g. directly behind the eye), or both. A magnetic field is generated by the magnet, the precise alignment of which can be controlled by placement of the magnet. For example, by placing the magnet behind the head and along the visual axis of the eye, the vector magnetic field may be directed precisely toward the center of the macula. Such a magnetic field causes the injected magnetic particles to migrate through the vitreous cavity and to concentrate at the region of interest, e.g. the macular region of the retina. As a result, the therapeutic agent is concentrated at the region of interest. This offers advantages in that a precise location within the eye may be targeted, making it possible to efficiently deliver the agent, to reduce the amount of therapeutic agent that is utilized, and to minimize exposure of the rest of the eye to the agent.

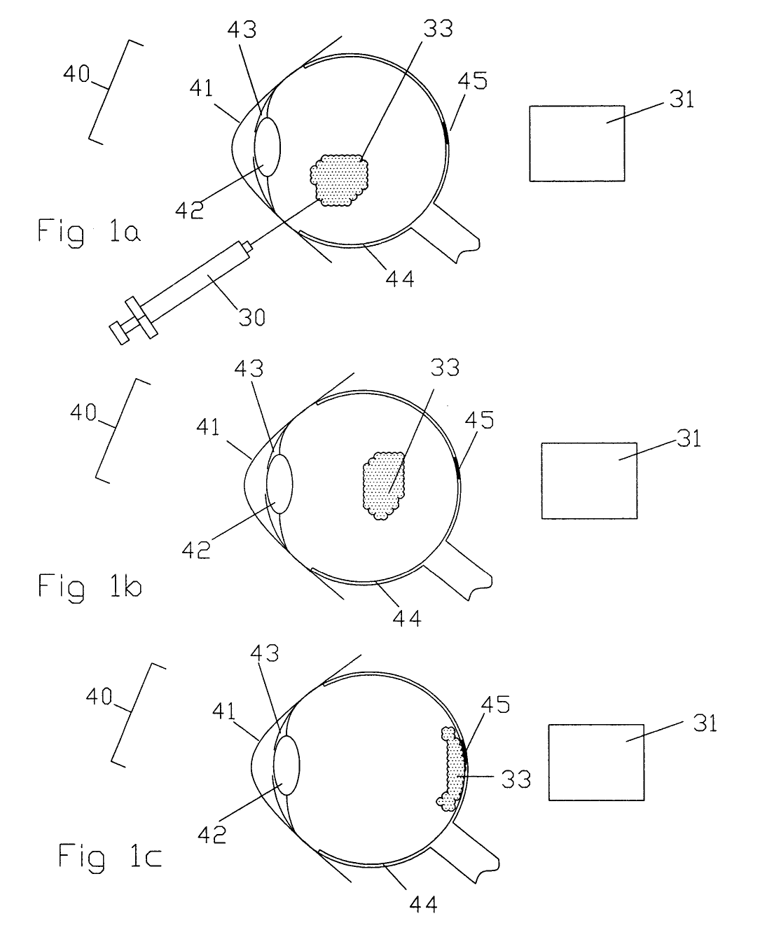

The invention may be further understood by reference to FIG. 1. FIG. 1 depicts a cross sectional view of an eye 40 showing the cornea 41 and lens 42, the vitreal cavity 43 and the retina 44. In FIG. 1a, syringe 30 containing magnetic particles with associated therapeutic agent(s) is used to inject the magnetic particles 33 into the vitreal cavity 43. Magnet 31 is oriented behind the eye so as to create a magnetic vector, the effect of which is to drive the magnetic particles 33 through the vitreal cavity 43, as illustrated in FIG. 1b, toward a desired target location within the eye, e.g. the macula 45 located at the back of the retina 44, as illustrated in FIG. 1c. FIG. 1c shows that the magnetic particles 33 have arrived at the macula 45, thus delivering the associated therapeutic agent(s) to the desired location.

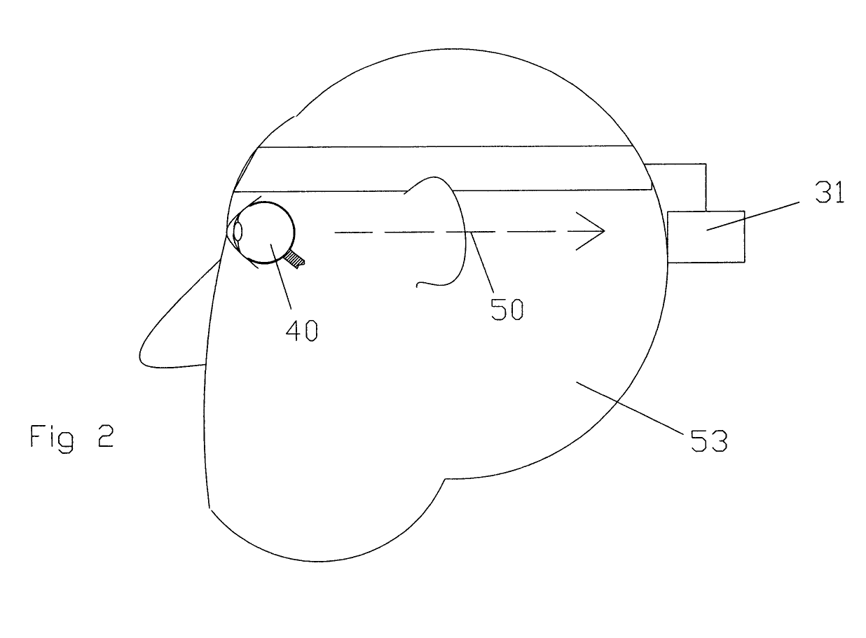

FIG. 2 further depicts the placement of magnet 31 with respect to the eye 40 of a patient 53 in order to generate magnetic vector 50. The force of the magnetic vector 50 drives the magnetic particles in the direction of the vector.

In some embodiments of the present invention, the location within the eye that is targeted for drug delivery is the macula. Macula targeting is useful for treatment of disorders peculiar to the macula, for example, exudative macular degeneration, and diabetic retinopathy. However, those of skill in the art will recognize that other areas accessible via the vitreous cavity also be targeted for the treatment of other conditions. For example, therapeutic agents may be targeted to other locations of the retina, or choroid for treating conditions such as ocular tumors.

The therapeutic agents that are delivered according to the present invention are attached to magnetic particles. By “attached to” we mean that the agent is chemically bonded, affixed, tethered, or otherwise associated with the particle by any of several means, including but not limited to: via magnetic, covalent, ionic, electrostatic, hydrophobic or hydrophillic interactions or attractions. Any means of association may be used so long as the agent is retained on the particle throughout the process of injection and positioning. Attachment may be by means of functional groups on the agent or on the magnetic particle, or both. In the process of linking an agent to a magnetic particle via a functional group, the functional group may be first attached to the magnetic particle and then the magnetic particle may be reacted with the agent to attach the agent to the magnetic particle. Alternatively, the agent itself may be derivatized so as to contain a functional group suitable for linking it to a magnetic particle. Some therapeutic agents may inherently possess a “functional group” (e.g. the sulfhydryl groups of cysteine residues, and the carboxy- and amino-terminal functional groups of polypeptides) that are suitable for reacting with a magnetic particle.

The association between the therapeutic agent and the magnetic particle may be by a direct association to the metal of the particle, or to an intervening layer or layers of molecules that coat the metal. In addition, the agent may be attached to a linker or spacer molecule (such as an alkyl chains or other polymer, examples of which are well known) is in turn attached directly to the metal, or to a molecular coating of the particle. Those of skill in the art will recognize that many strategies exist for coupling therapeutic agents to particles such as magnetic particles, and all such strategies are intended to be encompassed by the present invention.

Examples of agents which are suitable for use in the practice of the present invention include, but are not limited to drugs and small molecules, macromolecules such as proteins and fragments of proteins, peptides and polypeptides, antibodies, enzymes, nucleic acids such as DNA and RNA and DNA/RNA hybrids, saccharides, lipids, various hydrophobic or hydrophillic substances, lipophilic materials, enzymes, hormones, fibronectin, antibiotics, and the like. Further, such molecules and macromolecules may be naturally occurring or synthetic in nature. In preferred embodiments of the invention, the therapeutic agent is anti-VEGF (e.g. for the treatment of exudative macular degeneration) or a steroid (e.g. for the treatment of diabetic retinopathy).

In a preferred embodiment of the present invention, a single type of therapeutic agent is associated with the magnetic particle. However, those of skill in the art will recognize that this need not be the case. For example, it may be desirable to associate two (or more) therapeutic agents with a magnetic particle in order to achieve a desired result. Further, in some embodiments, magnetic particles with one associated therapeutic agent may be injected. However, in other embodiments two or more types of magnetic particles, each with differing attached agents may be injected simultaneously. Further, two or more types of magnetic particles with differing magnetic properties may be injected simultaneously. In this aspect of the invention, the particles which differ may be, for example, driven to different locations within the eye, or one may be driven to a location and another repelled from that or another location.

In yet other embodiments of the present invention, fluorescent or photoluminescent materials such as luminescent chromophores or dyes may be bound to the therapeutic agents, or to magnetic particles together with therapeutic agents, or on particles separate from the therapeutic agent. Such labels may be associated with the magnetic particles in order to aid in visual tracking of the therapeutic agent.

The magnetic particles with associated agent are injected in a formulation that includes a suitable physiological carrier such as saline. The preparation will be sterile and may also contain various other additives such as preservatives, buffering agents, colorants, and the like. In the formulation, the active ingredient will normally be present at about 1-99% of the total formulation, depending on the precise application. Further, the final concentration of therapeutic agent that is injected will vary depending on the agent itself and the disease or disorder that is being treated, as well as on such factors as the age, weight, and gender of the patient, or the progression of the disease. These variables will be well understood by and are best assessed by a skilled practitioner such as a physician. Due to the concentration of the therapeutic agent at the intended site of action, the amount of agent that is injected may be about 10-100 fold less, or alternatively about two-fold less, than that employed in current systemic or non-magnetically guided injections.

Several types of magnetic materials exist which are suitable for forming the magnetic particles used in the practice of the present invention. Examples include but are not limited to cobalt, magnetite, nickel, etc. In preferred embodiments, the material that is used is cobalt or magnitite. In the case of cobalt, a coating is used to prevent oxidation of the metal and loss of magnetic properties, i.e. to ensure magnetic stability. For example, the cobalt particles may be coated with a protective inert substance such as silica, and the therapeutic agent may be attached to the silica coating, either directly or via a linker or spacer molecule. The size of the magnetic particles for use in the practice of the present invention will be in the range of approximately 10⁻⁹ meters in diameter (i.e. nanoparticles), for example, in the range of 4 to 30 run, and preferably in the range of about 6-20 nanometers in diameter. The size of the magnetic microparticles for use in the practice of the present invention will be in the range of approximately 10⁻⁶ meters in diameter, for example, about 1 to 10 microns, and preferably in the range of about 2-4 microns in diameter. However, those of skill in the art will recognize that particles of a wide range of diameters may be employed in the present invention, e.g. from about 10⁻¹² meter to about 1 mm. Smaller particles are preferable as they move more readily through the medium with less disruption to the eye. The strength of the required magnetic field that drives the particles will vary, depending on, for example, particle size and composition of the particles.

Those of skill in the art will recognize that several strategies exist for producing magnetic particles for use in the practice of the present invention. Depending on the details of the procedure, the particles may be either nano- or micro-particles. For example, microparticles may be utilized with an external magnet and nanoparticles with an internal magnet. An example of nanoparticle construction is: a molecule containing two “tail” blocks of polydimethylsiloxane (PDMS) connected to a central “anchor” polymethylsiloxane with a cyano end group may be combined with cobalt octa-carbonyl in toluene, giving rise to PDMS coated cobalt nanoparticles. Reactive end groups can then be configured to bind therapeutic agents to the polymer/particle complexes. For example, anti-VEGF agent could be bound to the polymer/particle complex, and used to treat exudative age-related degeneration. Other synthesis schemes, including those for microparticles, are well known to those of skill in the art, for example those found in Harris et al., 2002; Rutnakornpituk et al, 2002a and 2002b, Stevenson et al., 2001; Philips et al., 1999; Wilson et al., 2002a and 2002b; and Connolly et al, 2002.

In the practice of the present invention, a formulation of magnetic particles with at least one associated therapeutic agent is injected into the eye of a patient in need of treatment with the therapeutic agent. In a preferred embodiment of the invention, the formulation is injected into the vitreous cavity of the eye. The details of carrying out such an injection, including, for example, the type and gauge of needle, the quantity of formulation, the duration of the injection, anesthetizing the eye prior to injection, and various precautions for patient safety, are known and are best determined by skilled practitioners such as physicians. See, for example: Eyetech Study Group, 2003; Gillies et al., 2003; Benhamou et al., 2003; Jonas et al., 2003; Martidis et al, 2002; Eyetech study group, 2002; Krzystokil et al., 2002.

Once the formulation has been injected into the eye, the patient is exposed to a magnetic field in order to cause the magnetic particles to migrate through the vitreous cavity to the desired position within the cavity, for example to the macula. When magnetic microparticles are employed, the magnetic field may be generated by an external magnet placed outside and behind the head at the level of the patient's eye. For example, placement of a magnet directly at the back of the head (and thus at about 100-mm behind the eye) can produce a magnetic field of about 0.5 Tesla, which is sufficient to drive magnetic microparticles through aqueous body fluids. However, those of skill in the art will recognize that the strength of the magnetic field to be employed in the present invention may vary from application to application but will generally be in the range of from about 0.001 to about 10 Tesla. Alternatively, the particles may be driven by a magnetic field generated by an “internal” magnet. By “internal” magnet, we mean that the magnetic field is generated by the placement of magnetic material into the body, for example into the eye itself or in the vicinity of the eye (e.g. directly behind the eye), and retained at the site of placement, perhaps permanently. Examples include injectable, polymerizable magnetic formulation which include cyanoacrylate and polymerize upon contact with water, forming a solid flexible magnetic mass that is retained at the site of injection.

In the case of external magnets, the patient is exposed to the magnetic field for a length of time that allows a sufficient quantity of magnetic particles (e.g. about 50 to 100%, or preferably 75-100%) to reach the intended location. Usually, the time of exposure to the magnetic field is in the range of about 10 to about 90 minutes, and preferably in the range of from about 10 to about 30 minutes. In the case of internal magnets, the time of exposure is moot since the magnetic field is permanent.

Tracking of the magnetic particles with either external or internal magnetic fields during this time may be accomplished by, for example, fluorescent tagging of the particles.

Treatments of this type may be given to a patient only once, or repeatedly at required intervals. For example, steroids are typically administered about every three months. Further, this treatment may be carried out in conjunction with other treatment protocols, such as systemic drug treatments (e.g. antibiotics) or various surgical procedures, as warranted for a specific situation.

### EXAMPLES

### Example 1

Under topical anesthesia, consisting of 0.5% tropicamide eyedrop, the sclera surface is sterilized with betadine solution, and 0.1 cc of polymer/microparticle/anti-VEGF solution (at a suitable therapeutic concentration) is injected via a 1 cc syringe with a 30 gauge needle 3.5 mm posterior to the cormeal limbus (see FIG. 3). The needle extends to the mid-vitrous of the eye of a patient and the material is injected. A magnetic field of about 0.5 Tesla is generated by placing a suitable magnet directly behind the head of the patient at the level of the eye. The magnet is kept in place for about 30 minutes (see FIG. 4) and the magnetic field drives the polymer/microparticle/anti-VEGF solution to the macula. Due to the small size of the particles, they pass into the subretinal space and the associated anti-VEGF inhibits the neovascularization associated with exudative macular degeneration.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### RFERENCES

Benhamou, N, Massin P, Haouchine B, Audren F, Tadayoni R, Gaudric A. 2003, Am. J. Ophthalmol., 135:246-9.

Connolly, J., T. G. St. Pierre, M. Rutnakornpituk and J. S. Riffle, “Silica Coating of Cobalt Nanoparticles Increases their Magnetic and Chemical Stability for Biomedical Applications,” European Cells and Materials, 3, Suppl. 2, www.eurocellmat.org.uk, 106-109 (2002).

Dailey J P, Phillips J P and Riffle J S 1999 J. Mag. Magn. Mat. 194 140

Dailey J P, Li C, Venkatesan V, Shi H, Taylor L, Davis S and Riffle J 1997 Investig. Ophthal. Vis. Sci. 38 3110

Eyetech Study Group, 2002, Retina, 22:143-52.

Eyetech Study Group, 2003, Opthalmology, 110:979-86.

Gillies G T, Ritter R C, Broaddus W C, Grady M S, Howard III M A and McNeil R G 1994 Rev. Sci. Instrum. 65 533

Gillies, M C, Simpson, J M, Luo W, Penfold, P, Hunyor A B, Chua, W, Mitchell, P, Billson, F. 2003, Arch. Ophthalmol., 121:667-73.

Häfeli U, Schütt W, Teller J and Zborowski M (eds.) 1997 Scientific and Clinical Applications of Magnetic Carriers (New York: Plenum)

Harris, L. A., J. D. Goff, A. Y. Carmichael, J. S. Riffle, J. J. Harburn, T. G. St. Pierre and M. Saunders, “Magnetite Nanoparticle Dispersions Stabilized with Triblock Copolymers,” Chemistry of Materials, 15(6), 1367-1377 (2003).

Jonas J B, Kressig I, Sofker A, Degenring R F. 2003, Arch. Ophthalmol., 121:57-61.

Lübbe A S, Alexiou C and Bergemann C 2001 J. Surg. Res. 95 200

Martidis A, Duker J S, Greenberg P B, Rogers A H, Puliafito C A, Reichel E, Baumal C. 2002. Ophthalmology, 109:920-7.

Mitchiner R, Kent T B, Peterson C and Rudge S R 2002, U.S. Pat. Ser. No. 6,488,615

Phillips, J. P., C. Li, J. P. Dailey, and J. S. Riffle, “Synthesis of Silicone Magnetic Fluids for Use in Eye Surgery,” J. of Magnetism and Magnetic Materials, Apr. 1, 1999, 140-148.

Rutnakornpituk, M., M. S. Thompson, L. A. Harris, K. E. Farmer, A. R Esker, J. S. Riffle, J. Connolly and T. G. St. Pierre, “Formation of cobalt nanoparticle dispersions in the presence of polysiloxane block copolymers,” Polymer, 43, 2337-2348 (2002a).

Rutnakornpituk, M., V. V. Baranauskas, J. S. Riffle, J. Connolly, T. G. St. Pierre and J. P. Dailey, “Polysiloxane Fluid Dispersions of Cobalt Nanoparticles in Silica Spheres for use in Ophthalmic Applications,” European Cells and Materials, 3, Suppl. 2, www.eurocellmat.org.uk, 102-105 (2002b).

\check{S}afa\check{r}ik I and \check{S}afa\check{r}ková M 2002 Monatsh. Chem. 133 737

Stevenson, J. P., M. Rutnakornpituk, M. Vadala, A. Esker, J. S. Riffle, S. W. Charles, S. Wells, and J. P. Dailey, “Magnetic Cobalt Dispersions in Poly(dimethylsiloxane) Fluids,” J. Magn. Magn. Maters., 225(1-2), 47-58 (2001).

Volkonsky V A, Dyuksherstnov S D, Chernyakov S V, Allen L M and Kent T B 2001 U.S. Pat. No. 6,200,547

Volkonsky V A, Dyuksherstnov S D, Chernaykov S V, Allen L M and Kent T B 2001, U.S. Pat. Ser. No. 6,482,436

Wilson, K. S., L. A. Harris, J. D. Goff, J. S. Riffle, and J. P. Dailey, “A Generalized Method for Magnetite Nanoparticle Steric Stabilization utilizing Block Copolymers Containing Carboxylic Acids,” European Cells and Materials, 3, Suppl. 2, www.eurocellmat.org.uk, 206-209 (2002b).

Wilson, K. S., M. Rutnakornpituk, L. A. Harria and J. S. Riffle, “Silicone magnetic fluids using poly(dimethylsiloxane)-b-poly(2-ethyl-2-oxazoline) as a steric stabilizer,” Polym. Prep., 43 (1), 732-733 (2002a).

We claim:

1. A method of delivering a therapeutic agent to a desired location within an eye, comprising the steps of
providing to said eye a formulation comprising magnetic particles, said magnetic particles having at least one associated therapeutic agent, and
using a magnetic field to move at least a portion of said magnetic particles to said desired location within said eye.

2. The method of claim 1 wherein said providing step is performed by injecting said formulation into the vitreous cavity of said eye.

3. The method of claim 1, wherein said desired location is a macula.

4. The method of claim 1 wherein said therapeutic agent is selected from the group consisting of anti-VEGF and a steroid.

7. The method of claim 1 wherein said magnetic particles are made from a material selected from the group consisting of cobalt, magnetite, and nickel.

8. The method of claim 1 wherein said particles are nanoparticles.

9. The method of claim 1, wherein said particles are microparticles.

10. The method of claim 1 wherein said portion is greater than 50% of the magnetic particles provided in said providing step.

11. The method of claim 1 wherein said magnetic particles have one associated therapeutic agent.

12. The method of claim 1 wherein said magnetic particles have more than one associated therapeutic agent.

13. The method of claim 1 wherein a label is associated with said magnetic particles.
